(19) United States
(12) Patent Application Publication
(10) Pub. No.: US 2015/0245948 A1
(43) Pub. Date: Sep. 3, 2015

(54) TECHNIQUE FOR TREATING PRESBYOPIA

(71) Applicant: WAVELIGHT GMBH, Erlangen (DE)

(72) Inventors: Theo Seiler, Eckental (CH); Christof Donitzky, Eckental (DE)

(21) Appl. No.: 14/428,629

(22) PCT Filed: Jul. 8, 2013
(86) PCT No.: PCT/EP2013/064399

§ 371 Date: Mar. 16, 2015

(51) Int. Cl. A61F 9/008; G05B 15/02

(52) U.S. Cl. CPC A61F 9/00808; G05B 15/02; A61F 2009/00895; A61F 2009/00897; A61F 2009/00882; A61F 2009/00872

(57) ABSTRACT

A system and method for reshaping a cornea of a patient's eye to treat presbyopia are disclosed as well as a method of generating a computer program for use in such system. Ray tracing is used to determine an ablation pattern that defines an amount of corneal tissue to be ablated, wherein the ablation pattern achieves a pre-defined amount of at least one of sphere, spherical aberration, coma, and astigmatism in the eye.

The present disclosure is concerned with techniques for reshaping a human cornea to treat presbyopia.

A frequently employed technique for eliminating visual defects of the human eye (such as e.g., myopia or hyperopia or astigmatism) is referred to as LASIK (Laser in-situ Keratomileusis). LASIK is a technique in which a small cover disc in the cornea is cut free, which cover disc remains connected to surrounding corneal tissue through a hinge, so that the cover disc can be folded aside to expose the underlying tissue regions of the cornea and can be folded back following an ablation process performed on the exposed tissue regions using UV laser radiation. The cover disc is conventionally referred to as a flap in the related art. On account of the removal of stromal material by the ablation process, a changed shape of the anterior surface of the cornea results after the flap has been folded back. The changed shape of the anterior corneal surface results in a different refractive behavior of the cornea and consequently of the overall imaging system of the eye. Suitable definition of the ablation pattern ensures that the initial visual defect is at least distinctly attenuated and, at best, is almost completely eliminated.

For purpose of this disclosure it is assumed hereinafter that the skilled reader is well-familiar with the LASIK technique.

Presbyopia is a well-known phenomenon due to aging. Presbyopia is caused by a loss of elasticity and/or by alterations of the core of the human lens such that the accommodation amplitude decreases with increasing age of the patient. This results in a loss of sharp vision in the near range.

According to the conventional art, presbyopia can be treated with the use of glasses having multifocal lenses (so-called multifocals). It is also known in the art to subject a presbyopic eye to a surgical operation to implant an intraocular lens (IOL) with multifocal characteristics.

In the field of ophthalmic refractive surgery, ray tracing is a technique that has attained increased attention in recent times. Ray tracing can be used to determine an ablation pattern defining an amount of corneal tissue to be removed in the course of a laser-surgical operation in order to reshape the anterior surface of the cornea for the purpose of reducing aberrations of the eye (visual defects). Ray tracing is usually based on an eye model (e.g., Gullstrand, Navarro, Liou and Brennan, Emsley et al.) in conjunction with measured data of the specific eye to be treated, wherein the measured data may relate to the anterior and/or posterior surface of the cornea, the depth of the anterior chamber of the eye, the dimension of the lens, the anterior and/or posterior surface of the lens, the length of the eye, data regarding the retina, and the measured wavefront of the entire eye.

An ablation pattern obtained through ray tracing is individual for the patient's eye and can be used to control a system for reshaping the cornea of the patient's eye wherein the system is controlled by a computer programmed accordingly.

Embodiments of the present disclosure are aimed at providing a technique for reshaping a cornea of a human patient's eye with the aim of treating presbyopia. In this context, treating presbyopia means that the treatment achieves an improvement for at least of one near and distance vision of the patient.

In one aspect, the present disclosure provides a method of determining a corneal ablation pattern for treating presbyopia of a human eye, comprising: defining a non-zero amount of induced aberration for a presbyopic human eye; and applying a ray tracing process to determine a corneal ablation pattern defining an amount of corneal tissue that, when removed from the eye, leaves the eye with the defined amount of induced aberration.

In another aspect, the present disclosure provides a method of treating presbyopia of a human eye, comprising: defining a non-zero amount of induced aberration for the eye; applying a ray tracing process to determine a corneal ablation pattern defining an amount of corneal tissue that, when removed from the eye, leaves the eye with the defined amount of induced aberration; and moving a focal point of laser radiation over an exposed surface of corneal tissue of the eye to remove corneal tissue in accordance with the determined ablation pattern.

In yet another aspect, the present disclosure provides a method of generating a control program for an ophthalmic laser apparatus, the control program containing instructions that, when executed by a control computer of the apparatus, control an optical system of the apparatus to move focused laser radiation over a target, the method comprising steps of: defining a non-zero amount of induced aberration for a presbyopic human eye; applying a ray tracing process to determine a corneal ablation pattern defining an amount of corneal tissue that, when removed from the eye, leaves the eye with the defined amount of induced aberration; and generating instructions for the computer program in accordance with the determined ablation pattern.

In still another aspect, the present disclosure provides a computer program product containing instructions that, when executed by a computer, perform a ray tracing process to determine a corneal ablation pattern defining an amount of corneal tissue that, when removed from a presbyopic human eye, leaves the eye with a pre-defined non-zero amount of induced aberration; and control a laser device to perform ablation on the eye in accordance with the determined ablation pattern.

In certain embodiments, the induced aberration includes at least one of sphere, spherical aberration, coma, and astigmatism. In certain embodiments, the induced aberration includes even higher-order aberration.

In certain embodiments, the ray tracing process is performed using measurement data for one or more of the following: pre-operative refraction of the eye, pre-operative aberration of the eye, corneal topography of the eye, one or more opto-geometrical parameters of the eye, one or more pupil-related parameters.

In embodiments of the present disclosure, the induced aberration is effective to increase a depth of field of the eye by about 1 to 3 diopters. According to a certain embodiment, the induced aberration is effective to increase a depth of field of the eye by about 2 diopters.

The present disclosure also encompasses a system for reshaping a cornea of a patient's eye to treat presbyopia, the system comprising a source for emitting laser radiation having radiation properties suitable for ablating corneal tissue from the eye; optical components for guiding and focussing the emitted laser radiation onto the cornea; and a computer for controlling the optical components in accordance with an ablation pattern defining an amount of corneal tissue to be ablated, wherein the ablation pattern is obtained on the basis of a predetermined amount of at least one of sphere, spherical aberration, coma, and astigmatism that the eye shall have after the eye has been ablatingly treated in accordance with the ablation pattern.

Induction of a certain amount of sphere, spherical aberration and/or coma and/or astigmatism means that, on purpose, the eye exhibits a longitudinal distribution of focal positions after the ablation process. Rather than defining an ablation pattern that results in a single focal point at or near the retina of the eye, embodiments of the invention achieves a focal range that is elongated in the direction of the optical axis of the eye, wherein in this range the focal dimensions are smaller than a pre-defined maximum value. This focal range may be referred to as a “focal tube”.

According to embodiments of the present disclosure, determine, an ablation pattern is determined for an individual patient's eye and ablation is then performed in according with the determined ablation pattern using a conventional LASIK technique. In this way, an optimum compromise can be achieved between visual acuity and depth of focus. This is achieved by intentionally inducing, through refractive laser surgery, specific amounts of aberration to the eye, wherein the aberration include at least sphere, spherical aberration, coma, and/or astigmatism. According to embodiments of the present invention, higher-order aberrations (HOA) may be induced in addition to the afore-mentioned types of lower-order aberration.

For example, the total visual comfort of the patient can be improved by accepting a loss in visual acuity of about 0.8 (visus) with regard to one eye and, at the same time, improve the depth of field by about 1-3 diopters. This can result in a total improvement of visual acuity (for both eyes) of about 40%.

According to embodiments of the invention, the dependency between visual acuity and depth of focus is utilized such that a well-defined certain amount of sphere, spherical aberration and/or coma (optionally also using HOA) is maintained in order to treat presbyopia, i.e. to obtain an increased depth of focus and, in particular, improved vision in the near range. The calculated ablation pattern may account for potential future changes of the amount of presbyopia of the pertinent eye.

Also, the mutual correlation between coma and astigmatism and/or sphere and spherical aberration can be used because they often compensate each other.

The determination of the ablation pattern and, thereby, generation of the focus tube is performed as follows. Based on parameters of the individual eye of the patient, as outlined above, an individual model of the patient's eye is generated. This procedure is known in the art. A focus tube of selected diameter and length is introduced into the individual eye model. The ray tracing technique is used to define the focus tube on the basis of the selection of a sphere, spherical aberration, coma, and/or astigmatism. The parameters are, by calculation, changed until a suitable focus tube length and focus tube diameter is obtained which results in a wanted depth of field.

In a normal eye, ideally, the light is bundled such that a sharp image is generated in the layer of photo-receptors of the retina. Geometrically, therefore, the focus must be located directly in front of that layer and, during accommodation, the refractive power is moved back and forth to bring objects in various distances in focus.

The human eye usually applies two different mechanisms for accommodation in the near field, namely the accommodation and the pseudo accommodation. Accommodation is an active change of the form of the lens. The ability of the lens to deform decreases during lifetime to typically 0 diopters at an age of about 50 years. The pseudo accommodation results from pin hole effects (depth of field) but is also caused by High Order Aberrations (HOA) in the cornea and in the lens. By fixing an object in the near field the pupil becomes smaller and in connection with spherical aberrations (prolate cornea, old lens) the eye becomes somewhat near-sighted and thus the patient achieves improved visual acuity in the near field. The effect obtained by embodiments of the present invention can be understood in the sense of pseudo accommodation. The improved accommodation is in the range of about 1 to 3 diopters. Based on pre-measured optical aberrations, when indicated including HOA, of an eye of a patient purposely an ablation pattern is calculated by ray tracing and mixing certain amounts of sphere, spherical aberrations, astigmatism, and coma such that a compromise can be achieved that is agreed upon by the patient. That compromise is achieved by improved pseudo accommodation by the expense of a somewhat reduced visual acuity (visus). The optimum compromise can be achieved, in special cases, by using adaptive optics in order to test the individual patient's perception.

Once the ablation pattern is calculated as described above, the patient's eye can be treated according to well-known LASIK-technologies or PRK.

According to an embodiment of the invention, the ablation pattern is determined also on the basis of the following measured parameters, namely the pre-operative refraction (sphere and cylinder), the corneal tomography (using, e.g., a WaveLight Oculyzer), the pre-operative aberration (using e.g., a WaveLight Analyzer), geometrical data of the eye (length of bulbous, thickness of the lens), data of the pupil (mesopic and photopic pupil, near reflex).

By using the afore-described technology, a platform is provided that can be used by the ophthalmologist and the patient and by using pre-operatively adaptive optics, an optimum compromise between depth of focus (in diopters) and a certain loss of visual acuity or distance. To achieve about two diopters of pseudo accommodation the “length” of the focus tube (Rayleigh length) is about 1.2 mm.

Adaptive optics for studying visual functions are described in a paper of Austin Roorda, Journal of Vision (2011) 11(5): 6, 1-21.

Visual acuity under combined astigmatism and coma are studied by de Gracia, P., Dorronsoro, C., Marin, G., Hernandez, M., & Marcos, S. (2011). Visual acuity under combined astigmatism and coma: Optical and neural adaptation effects. Journal of Vision, 11(2): 5, 1-11.

Rocha, Vabre, Chateau, and Krueger describe expanding depth of focus by modifying higher-order aberrations induced by an adaptive optics visual simulator in J Cataract Refract Surg 2009; 35: 1885-1892.

In the following a system and method of the invention are described with regard to embodiments shown in the figures.

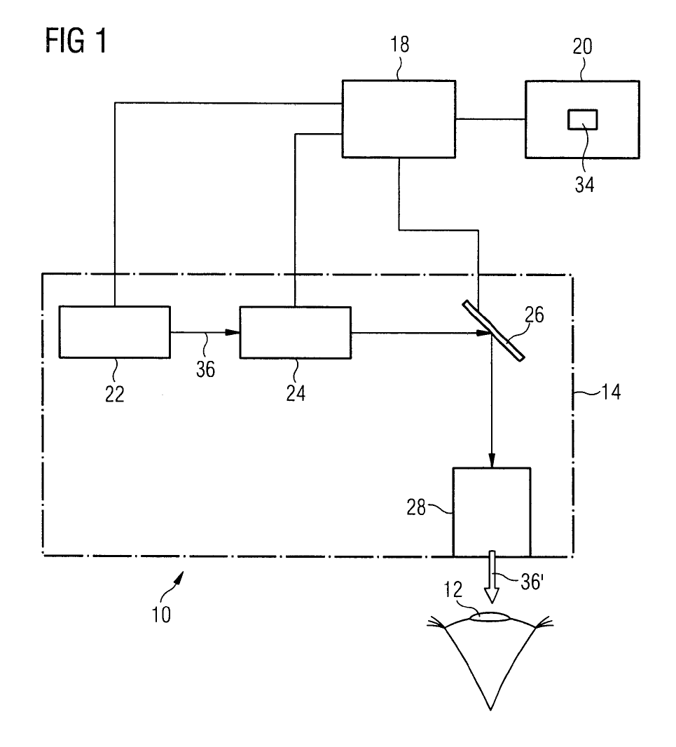

FIG. 1 shows, schematically, a system for reshaping a cornea;

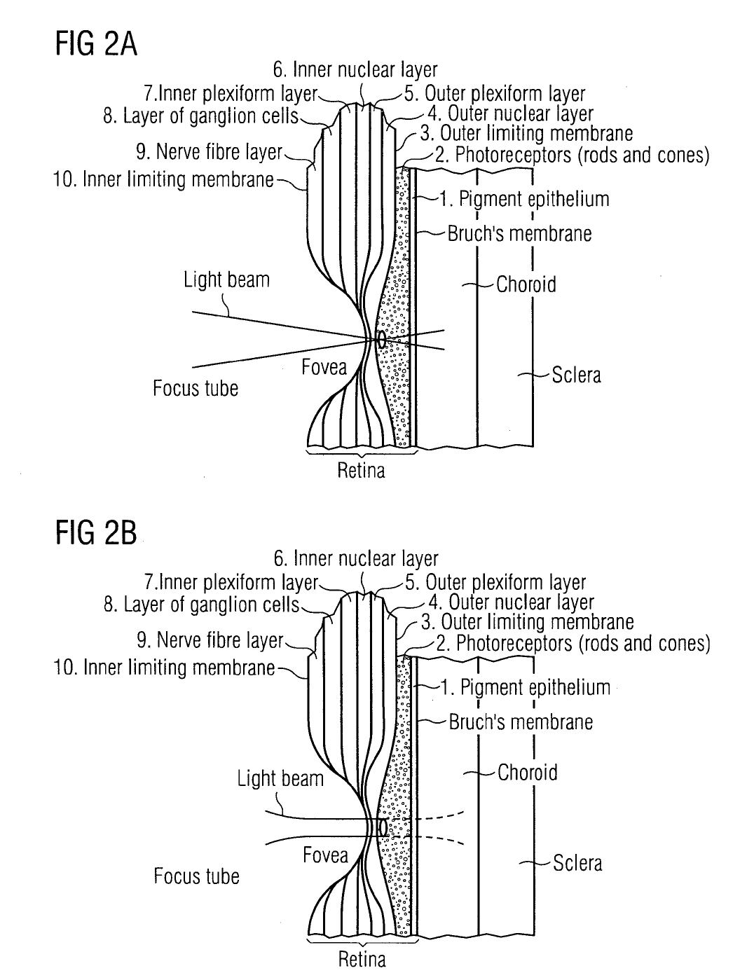

FIG. 2A shows, in expanded presentation, the focussing of light within the human eye near the retina; and

FIG. 2B shows the focus tube achieved in accordance with embodiments of the

According to FIG. 1, a system 10 for reshaping a cornea 12 of a patient's eye to treat presbyopia comprises a laser device 14, a control computer 18, and a memory 20 coupled as exemplary shown. The laser device 14 includes a laser source 22, a scanner 24, one or more optical mirrors 26, and a focussing objective 28 coupled as exemplary shown. The memory 20 stores a computer program for controlling optical means, in particular the laser source 22, the scanner 24, one or more optical mirrors 26, and the focusing objective 28.

The laser source 22 generates laser radiation 36 which, under control by computer 18, is guided and focussed onto the cornea 12 of the eye. The scanning is performed in accordance e.g., with LASIK technology in order to ablate corneal tissue to reshape the cornea. An ablation pattern is stored in program 34 and the computer 18 controls the focused laser radiation 36′ onto the eye in accordance with the ablation pattern to treat, in the present case, presbyopia. The laser source is, typically, an excimer laser.

The computer program 34 stored in memory 20 comprises, inter alia, an ablation pattern defining the corneal tissue to be ablated (removed) from the cornea 12.

The ablation pattern is generated by first measuring at least the following parameters of the eye to be treated: pre-operative refraction (sphere, cylinder), corneal tomography, using e.g., an WaveLight Oculyzer, pre-operative aberrations, using e.g., an WaveLight Analyser, geometrical data of the eye, in particular the length of the bulbous, the thickness of the lens, and data regarding the pupil, in particular data regarding the mesopical and the photopical pupil, and regarding the reflex in the near field.

Based on such measured data, as is principally known in the art, by ray tracing technique, an ablation pattern is calculated for the individual eye of the patient. According to embodiments of the invention, only one eye of the patient is treated as described here, whereas the other eye may be treated in a different manner.

The ablation pattern, in accordance with this embodiment, is not calculated such that the final properties of the eye achieved after the refractive surgery are as close as possible to an ideal eye. Rather, purposely, the ablation pattern is calculated such that certain aberrations remain in the cornea as outlined above. The ablation pattern is such that, after ablation, the optical properties of the eye comprise a predetermined amount of sphere and/or spherical aberration and/or coma, and/or astigmatism. Depending on the measured data of the eye, the ablation pattern can be calculated such that higher-order aberrations (HOA) also remain after surgery. The so-calculated ablation pattern is used by the system of FIG. 1 to effect ablation of the cornea 12.

FIGS. 2A and 2B illustrate the effect achieved when applying the afore-described methods of inducing a predetermined amount of aberration into a presbyopic human eye. FIG. 2A shows the focussing of light within the eye near the retina without the induced “focal tube” as described above. FIG. 2B shows the induced “focal tube” in accordance with the above-described embodiments of the present invention. The anatomical details are indicated in the figures.

1. A method of determining a corneal ablation pattern for treating presbyopia of a human eye, comprising:
defining a non-zero amount of induced aberration for a presbyopic human eye; and
applying a ray tracing process to determine a corneal ablation pattern defining an amount of corneal tissue that, when removed from the eye, leaves the eye with the defined amount of induced aberration.

2. The method of claim 1, wherein the induced aberration includes an aberration selected from a group consisting of sphere, spherical aberration, coma, astigmatism, or a combination of at least two aberrations.

3. The method of claim 2, wherein the induced aberration further includes a higher-order aberration.

4. The method of claim 1, wherein the ray tracing process is performed using measurement data for one or more of the following: pre-operative refraction of the eye, pre-operative aberrations of the eye, corneal topography of the eye, one or more opto-geometrical parameters of the eye, one or more pupil-related parameters.

5. The method of claim 1, wherein the induced aberration is effective to increase a depth of focus of the eye by about 1 to 3 diopters.

6. A method of treating presbyopia of a human eye, comprising:
defining a non-zero amount of induced aberration for the eye;
applying a ray tracing process to determine a corneal ablation pattern defining an amount of corneal tissue that, when removed from the eye, leaves the eye with the defined amount of induced aberration; and
moving a focal point of laser radiation over an exposed surface of corneal tissue of the eye to remove corneal tissue in accordance with the determined ablation pattern.

7. A method of generating a control program for an ophthalmic laser apparatus, the control program containing instructions that, when executed by a control computer of the apparatus, control an optical system of the apparatus to move focused laser radiation over a target, the method comprising steps of:
defining a non-zero amount of induced aberration for a presbyopic human eye;
applying a ray tracing process to determine a corneal ablation pattern defining an amount of corneal tissue that, when removed from the eye, leaves the eye with the defined amount of induced aberration; and
generating instructions for the computer program in accordance with the determined ablation pattern.

8. A computer program product containing instructions that, when executed by a computer,
perform a ray tracing process to determine a corneal ablation pattern defining an amount of corneal tissue that, when removed from a presbyopic human eye, leaves the eye with a pre-defined non-zero amount of induced aberration; and
control a laser device to perform ablation on the eye in accordance with the determined ablation pattern.

9. A system for treating presbyopia, comprising:
a laser device configured to generate laser radiation;
a control computer configured to:
perform a ray tracing process to determine a corneal ablation pattern defining an amount of corneal tissue that, when removed from a presbyopic human eye, leaves the eye with a pre-defined non-zero amount of induced aberration; and
control the laser device to perform ablation on the eye with the laser radiation in accordance with the determined ablation pattern.
